(19) United States
(12) Patent Application Publication
(10) Pub. No.: US 2015/0191771 A1
(43) Pub. Date: Jul. 9, 2015

(54) EMBRYO SAMPLING METHOD

(71) Applicant: Syngenta Participations AG, Basel (CH)

(72) Inventors: William Paul BULLOCK, Slater, IA (US); Ulich Stephan HANNAPPEL, Slater, IA (US); Katie Marie HUNTER, Slater, IA (US); Christopher Cody HEJLIK, Slater, IA (US)

(73) Assignee: Syngenta Participations AG, Basel (CH)

(21) Appl. No.: 14/150,938

(22) Filed: Jan. 9, 2014

(51) Int. Cl. C12Q 1/68; A01H 4/00; A01H 1/04; A01H 5/10

(52) U.S. Cl. CPC C12Q 1/6806; A01H 5/10; A01H 4/008; A01H 1/04

(57) ABSTRACT

The present invention belongs to the field of sampling of biological materials. In particular, it relates to tissue or cellular content extraction from immature corn embryos while retaining the viability of said embryos.

### INTRODUCTION

Maize plant breeding has increasingly become more dependent upon a) doubled haploid (DH) processes to rapidly create true breeding inbred parental lines, b) the consolidation of desired quantitative trait loci into individual corn lines, and c) the introgression of transgenes into desired commercial inbreds. With respect to these goals, one of the challenges in commercial plant breeding is the rapid acceleration of the above described breeding process. In particular, the inability to make breeding or genetic selections based on plant genotype or biochemical constitution/makeup soon after seed development imposes time, resource and consumable costs and constraints. For example, the postponement of breeding advancement or other selection decisions creates significant management challenges, because large populations of individual seeds and plants must be carried forward due to the inability to make data driven selection decisions earlier in the breeding pipeline.

This present application illustrates methods which 1) provide for very early identification of genetically or compositionally selected individuals and 2) sustains the viability/germination capacity of these individuals. More specifically, scutellar and/or embryo axis tissue or cellular contents derived from immature corn (about 5 to 24 days post pollination, the range of which is partially dependent upon environmental growing conditions) embryos are sampled to determine their genotype or biochemical composition. Post tissue sampling, the resulting embryos are quite capable of germinating and developing into normal corn plants.

The literature alludes to DNA extraction from embryos, but do not indicate how this might effectively be done. For example, Jordano & Godoy, in “Lab protocols for DNA extraction and genotyping of SSR microsatellites for Prunus mahaleb (Rosaceae) seeds and Quercus acorns” (2005) describe the extraction of DNA from entire embryos and so no viability is retained. Sparks et al., Plant Physiology December vol. 133 no. 4 1809-1819 (2003) also describe DNA extraction from embryos. Again, viability is not retained. The Pioneer patent application (WO2011/019863 A1) discusses automated embryo extraction from corn seeds. However, it does not claim or describe “retained viability” of the embryos after tissue removal. The patent does not describe any specific method as to how to isolate cells from embryos. Monsanto patent (U.S. Pat. No. 8,312,672) describes the problem, but proposes to analyze endosperm tissue (via seed chipping) to fingerprint the embryo.

The tissue sampling and cellular content sampling processes described below provide for 1) early acquisition of genetic and biochemical data from which to make data driven breeding decisions, 2) processes for tissue and cellular content sampling, 3) minimization of requisite DNA purification steps, 4) reduced cost per DNA extraction, 5) allows for the direct use of the resulting DNA for PCR analysis, 6) a viability retaining tissue sampling and cellular content sampling method targeting very immature plant tissues, and 6) methods which are readily automated.

The methodology that the inventors of the present application have developed illustrates how DNA extraction might effectively be done. The methods can be categorized by extraction methodology: 1) mild chemical disruption of cells/organized tissues such that DNA and other cellular content is eluted from some embryo tissue into the surrounding solution and 2) mechanical means which remove tissue and/or cellular contents from the scutellum of immature corn embryos. Uniquely, each of these extraction methods provides for viability-retaining germination capacity within the sampled immature embryos.

In general terms, the methodology includes a series of steps: 1) Immature corn embryo extraction, 2) Tissue/DNA/cellular content extraction by either a) soaking of an embryo in solution that provides for DNA and cellular content leakage from cells/organized tissues, or b) sampling of tissue from immature scutellum by plugging, or penetrations or abrasion or scraping, or slicing 3) the option for automation of the aforementioned processes, and 4) high rates of embryo germination. As a series of steps, the inventors provide various methods for a viability-retaining tissue/DNA and cellular content extraction processes that provides the opportunity to make genetic data driven selection decisions in one of the earliest possible manners.

### BRIEF DESCRIPTION OF THE FIGURES

FIG. 1 shows results of allele identification from individual haploid corn embryos post soaking for 1 hour in 5 mM NaOH.

FIG. 2 shows results of allele identification from individual haploid corn embryos post soaking for 1 hour in 10 mM NaOH.

FIG. 3 shows results of allele identification from individual haploid corn embryos post use of endosperm wash removal method.

FIG. 4 shows results of allele identification from individual haploid corn embryos post use of endosperm wash removal method 2.

FIG. 5 shows results of allele identification from individual diploid corn embryos soaked in 20 mM NaOH for 1 hour.

FIG. 6 shows results of allele identification from individual diploid corn embryos as a function of scutellar plug tissue sampling via the use of a small bore pipette tip.

FIG. 7 shows results of allele identification from 288 individual haploid corn embryos as a function of scutellar plug tissue sampling via the use of a small bore pipette tip.

FIG. 8 shows results of allele identification from individual haploid corn embryos as a function of scutellar tissue sampling via toothpick penetration.

FIG. 9 shows results of allele identification from individual diploid corn embryos as a function of scutellar tissue sampling via toothpick penetration.

FIG. 10 shows photographs of haploid seedlings resulting from the soaking of haploid embryos in NaOH.

### SCOPE OF THE INVENTION

There is provided a non-destructive, viability retaining method of sampling a plant embryo, comprising the step of removing a tissue sample comprising at least a portion of the scutellum region of the embryo. In one embodiment, the tissue sample derives from the embryonic axis. In one embodiment, the tissue sample derives from the scutellum and embryonic axis.

There is also provided a non-destructive, viability retaining method of sampling a plant embryo, comprising the step of releasing and collecting cells or cellular contents from the scutellum region of the embryo. In one embodiment, the cells or cellular contents are from the embryonic axis region of the embryo.

In one embodiment, the method according to the invention further comprises the step of performing analysis on the tissue sample or cellular contents from the embryo.

In one embodiment, the method according to the invention further comprises the step of selecting an embryo based on the result of the analysis.

In one embodiment, the method according to the invention is at least semi-automated. In one embodiment, the method according to the invention is automated.

In one embodiment of the method according to the invention, the embryo is at the non-mature stage of development.

In one embodiment of the method according to the invention, the sample removed comprises tissue from the coleoptilar end of the embryo.

In one embodiment of the method according to the invention, the sample removed comprises tissue from the non-coleoptilar end of the embryo.

In one embodiment of the method according to the invention, the sample removed comprises tissue from the equator or middle region of the embryo.

In one embodiment of the method according to the invention, the sample is removed using a penetrant. In one embodiment, the penetrant is a toothpick. In one embodiment, the penetrant is a pipette tip. In one embodiment, the penetrant is a solid needle. In one embodiment, the penetrant is a hollow needle. In one embodiment, the penetrant is a capillary tube.

In one embodiment, the sample is removed using a charged penetrant which attracts or retains tissues and/or cellular content e.g. DNA. In one embodiment, the penetrant may have a roughened surface or a smooth surface.

In one embodiment of the method according to the invention, cells and/or cellular contents are removed using sandpaper, or similar abrasive material in order to release cells and cellular contents and optimize their retention on the abrasive material.

In one embodiment of the method according to the invention, the sample is removed by cell disruption using a liquid solution to bathe the embryo for a sustained period of time. In one embodiment, the liquid solution is NaOH

In one embodiment of the method according to the invention, the sample is removed by cell disruption using gentle sonication and optionally, cell wall degrading enzymes.

In one embodiment, the method according to the invention further comprises the collection of cells and cellular contents from the liquid.

In one embodiment of the method according to the invention, the sample is removed by slicing with a knife or scalpel or a laser or similar devices.

In one embodiment of the method according to the invention, the removed sample is subjected to genetic, chemical or biochemical analysis.

In one embodiment of the method according to the invention, the embryo is a maize embryo. In one embodiment of the method according to the invention, the embryo is a wheat embryo.

In one embodiment of the method according to the invention, the embryo is a rice embryo.

The concept could be applied to somatic embryos derived from microspore culture.

In one embodiment, the embryo is a microspore derived embryo from a Brassica species. In one embodiment, the embryo is a microspore derived embryo from a Hordeum species. In one embodiment, the embryo is a microspore derived embryo from a Cucumis species. In one embodiment, the embryo is a microspore derived embryo from a Zea Mays species. In one embodiment, the embryo is a microspore derived embryo from a Triticum species. In one embodiment, the embryo is a microspore derived embryo from a Capsicum species. In one embodiment, the embryo is a microspore derived embryo from an Oryza species. In one embodiment, the embryo is a microspore derived embryo from a Saccharum species.

There is also provided a plant embryo, wherein said embryo has been sampled according to the method of the invention.

There is also provided the use of a plant embryo according to the invention in plant production.

There is also provided a maize plant produced from an embryo according to the invention.

There is also provided a method of data driven plant advancement, comprising the steps of a) inducing haploids; b) extracting and identifying haploid embryos from amongst a mixed population of diploid and haploid embryos; c) obtaining a tissue sample, cells or cellular contents from the extracted embryos in a non-destructive manner, said samples preferably comprising the scutellum region; and d) doubling chromosome number; wherein step c) can take place before and/or after step d).

In one embodiment, the method of the invention further comprises one or more of the steps of e) analyzing the genetic, chemical or biochemical characteristics of the sampled embryos; f) advancing selected embryos in a breeding process based on the analyzed characteristics; and g) producing seedlings from the selected embryos. There is also provided a method of data driven plant advancement, comprising the steps of h) obtaining a tissue sample, cells or cellular contents from the extracted diploid embryos in a non-destructive manner, said samples could be any cells but preferably comprising the scutellum region i) analyzing the genetic, chemical or biochemical characteristics of the sampled embryos; j) advancing selected embryos in a breeding process based on the analyzed characteristics; and k) producing seedlings from the selected embryos.

In one embodiment, the method of the invention further comprises the production of seeds from the seedlings of steps g) and k)

In one embodiment of the method according to the invention, the embryos are put into stasis storage to achieve a quiescent condition until the outcome of the genetic, chemical or biochemical analysis is known.

In one embodiment of the method according to the invention, the embryos are stored in a controlled atmosphere. In one embodiment of the method according to the invention, the embryos are stored at a controlled temperature. In one embodiment of the method according to the invention, the embryos are stored in a controlled atmosphere and temperature.

In one embodiment, stasis storage controls cell division in a productive fashion to the benefit of chromosome doubling. This equates to better fertility and seed production.

In one embodiment of the method according to the invention, the embryos are stored in the presence of anti-ethylene or ethylene metabolism impacting compounds which reduce the production of, or bind with and remove, or obviate the impact ethylene production. In one embodiment, these are compounds which bind to the same receptor sites to which ethylene also binds. In one embodiment, the compound is silver nitrate. In one embodiment, the compound is silverthiosulfate. In one embodiment, the compound is 1-MCP. In one embodiment, the compound is aminoethoxyvinylglycine. In one embodiment, the compound is 2,5-Norbornadiene. In one embodiment, potassium permanganate is used to bind gaseous ethylene. In one embodiment, the compound is an anti-ethylene or ethylene metabolism impacting compounds known to the skilled person which reduce the production of, or bind with and remove, or obviate the impact ethylene production.

In one embodiment of the method according to the invention, the embryo is manipulated using a robotic arm system; a camera is used to select haploids or non-haploids; and a camera or vision system is used to guide the sampling instrument to the tissue to be sampled.

There is also provided a method of haploid embryo discrimination, comprising the steps of capturing an image of a population of embryos; identifying haploid embryos in said population; moving selected embryos via transporting means.

In one embodiment, the method of the invention further comprises the steps of transferring all embryos or selected embryos to a different location using said transporting means.

In one embodiment, the method of the invention further comprises the steps of placing the attached embryos such that tissue sample(s) can be removed from the embryos; transferring all embryos or selected embryos to a different location. In one embodiment, the location is a storage location, preferably stasis storage.

In one embodiment, the method of the invention further comprises the steps of placing the attached embryos such that a tissue sample can be removed from the embryos; transferring all embryos or selected embryos to a controlled atmosphere and temperature environment suitable for embryo storage.

In one embodiment, the embryo is moved so that it is contacting a solution so that the cells can be disrupted and the cellular content can be collected.

In one embodiment of the invention, the embryos are identified using algorithms based on either color or spectral analysis.

In one embodiment of the invention, the method is at least semi-automated.

In one embodiment of the invention, the tissue sampling steps are automated.

### DEFINITIONS

The technical terms and expressions used within the scope of this application are generally to be given the meaning commonly applied to them in the pertinent art of sampling biological materials if not otherwise indicated herein below.

Zygotic Embryo: An embryo composed of a central embryo axis and scutellum.

Embryo Germination: The growth and extrusion of root and shoot axes from the immature embryo.

Microspore Derived Embryo: An embryo derived from an embryogenic developmental process initiated from a plant microspore.

Scutellum: A scutellum is a modified cotyledon and is a component part of the monocotyledoneus embryo. It is attached to the embryo axis in the scutellar node. It acts as a food storage organ and serves to digest and transport the endosperm nutrients during germination.

Embryo axis: Embryo axis is formed by the primary root, protected by the coleorhiza, and leaf primordia (plumule), protected by the coleoptile.

Coleoptile: The first true leaf and is modified to act as a protective covering for the plumule or first bud of the plant and grows throughout the soil during germination.

Immature Embryo: An embryo which has not yet undergone a desiccation process and has not achieved an associated quiescent state. Immature embryos can be extracted from the seed or caryopsis either by hand or via automation within a matter of days post pollination and which under the appropriate conditions are capable of germination.

Mature Embryo: A corn embryo which has undergone the desiccation process and achieved an associated quiescent state.

Scutellum: Coleoptilar End. The area or region of the scutellum which surrounds or is adjacent to the coleoptile.

Scutellum: Non-Coleoptilar End. The area or region of the scutellum which surrounds or is adjacent to the coleorhiza.

Embryonic Axis: The embryo axis is formed by the primary root, protected by the coleorhiza, and the stem tip, protected by the coleoptile.

Tissue Sample: A piece of tissue which is removed from an immature zygotic or microspore-derived embryo.

Plugging: Removal of plug tissue from an embryo.

Cellular Contents: Internal constituents of a plant cell which may include DNA or other cytoplasmic components such as organelles, protein, oils, sugars, starch or other organic compounds.

Penetrant: A physical structure which is capable of penetrating into plant tissue. The penetrant may be solid or hollow. It may contain a smooth or abrasive or charged surface to facilitate the removal of tissue and cellular contents from plant embryos.

Tissue Plug: A tissue sample collected via the penetration of embryo tissue by a hollow penetrant.

Abrasive Surface: A surface which when rubbed or placed in contact with the desired plant surface is capable of abrading the zygotic or microspore derived embryo so as to remove a tissue sample.

Haploid Induction: A plant process whereby the creation of haploid maize zygotic embryos occurs. In maize, various haploid inducing lines (e.g. RWK, RWS, Stock 6, MHI, etc.) can be used as male parents to pollinate other corn germplasm from which maternal haploid embryos are produced. In Wheat, corn pollen is used to stimulate haploid wheat embryo development.

Viability Retaining: Describes the circumstance wherein a plant embryo remains viable and capable of germination post removal of tissue (tissue sampling and/or the disruption of cells).

Non-destructive manner: A method of tissue sampling and/or the disruption of cells which provides for the viability and germination capacity of an immature zygotic or microspore derived embryo.

Storage of Embryos: Storage of immature zygotic or microspore derived zygotic embryos in conditions which minimize respiration and/or metabolic activity in order to maintain the embryos in a viable-quiescent condition.

Stasis Storage: Storage of a plant embryo in a quiescent condition.

Quiescent Condition: A state of suspended growth or resting condition of the embryo in which metabolic activity is substantially slowed.

Controlled Atmosphere: An atmospheric composition(s) which reduces or minimizes respiration and/or metabolic activity so as to provide for the storage of immature zygotic or microspore derived embryos in a quiescent condition.

Haploid Embryo: This references the genetic ploidy of a zygotic or microspore derived embryo and more specifically said embryos contains half the normal number of chromosomes.

Doubled Haploid Embryo: A zygotic or microspore derived embryo to which a chromosome doubling process has been applied. The embryo may consist entirely or partially of cells which contain a diploid number of chromosomes.

Automated: A process or process step or series of process steps which is or are done entirely by machine.

Semi-automated: A process or process step or series of process steps which is or are done in part by machine and in part by a human being.

Robotic Arm System: A computer controlled electro-mechanical device with multiple degrees of freedom which provides for a simulation of the capabilities of a human arm and hand such that embryos can be moved from one location to another. Furthermore, the robotic arm system provides a means for the taking of a tissue sample from previously describe embryos and transport of said tissue sample(s) and their placement in second location.

Vision system: A camera system capable of distinguishing human visible or multispectral characteristic of zygotic or microspore derived plant embryos. The aforementioned camera system provides information to a computer controlled device such as a robotic arm to facilitate automated process steps.

Transporting Means: This refers to the use of a robotic arm or other mechanical device which is used to move zygotic or microspore derived embryos from one location to another.

Algorithms: A process or set of rules to be followed in calculations or other problem-solving operations, especially by a computer.

Data Driven Plant Advancement/Selection Decision: Use of phenotypic or genetic or biochemical data derived from an individual or groups of individuals or populations in order to select individual(s) or groups of individuals or populations for continued breeding or genetic enhancement efforts or further examination.

As used in this specification and the appended claims, the singular forms “a”, “an”, and “the” include plural referents unless the context clearly dictates otherwise. Thus, for example, reference to “a plant” includes one or more plants.

As used herein, the term “about” when referring to a value/amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, the term “breeding”, and grammatical variants thereof, refer to any process that generates a progeny individual. Breeding can be sexual or asexual, or any combination thereof. Exemplary non-limiting types of breeding include crossings, self-pollination, doubled haploid derivative generation, polyploidization and combinations thereof.

A “plant” is any plant at any stage of development.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

As used herein, the term “progeny” refers to the descendant(s) of a particular cross or individual. Typically, progeny result from breeding of two individuals, although some species (particularly some plants) can be self-pollinated (i.e. the same plant acts as the donor of both male and female gametes). The descendant(s) can be, for example, of the F1, the F2, or any subsequent generation.

### EXAMPLES

Below are provided examples of 1) chemical elution of DNA from immature corn embryos, 2) mechanical methods of tissue extraction from scutellar tissue of immature corn embryos, 3) PCR-based genotyping results using the extracted DNA from individuals, and 4) germination capacity of embryos having already received the aforementioned chemical or mechanical treatments.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### Example 1

### Ear Sterilization and Embryo Rescue

Ears of corn of varying ages (about 15 to 24 days post pollination) are sterilized in a 50% bleach solution and then rinsed with sterile water. Kernel caps are removed with a scalpel and immature zygotic embryos of various sizes [about 4-6 mm in length] are extracted by hand and placed either into a petri dish containing a filter paper saturated with Murashige and Skoog (M.S., 1962) based liquid growth medium or directly onto the surface of M.S. based solidified growth medium. The embryos are then cultured under grow lights (16 hr. day length, 400 umol, 31 C/87 F day 22 C/72 F night).

Diploid embryos may be used directly for tissue extraction or cell disruption. Alternatively and when a scutellar pigmentation marker such as R1scm2 or R1scm3 or R1scm122, or R1scm124 are employed, a short period of time is provided to allow for the discrimination of haploid from diploid embryos. Diploid or haploid embryos are then provided for tissue sampling or cell disruption as described below.

### Example 2

### Evaluation of the Germination Capacity of Haploid and Diploid Immature Corn Embryos

Control or tissue sampled haploid and diploid immature corn embryos are placed onto the surface of solidified M.S. based growth medium and cultured for under the following growing conditions: 16 hr. day length, 400 umol PAR light, 31 C/87 F day 22 C/72 F night). Embryo germination is scored after a period of 3 to 5 days.

### Example 3

### Soaking of Haploid Embryos in NaOH to Facilitate DNA Extraction

Experiment 1. For the removal of adherent endosperm tissue, all embryos were pooled and transferred into a 50 ml tube containing 40 ml MS buffer and 3% sucrose. To enhance the washing efficiency, the tube was placed on a horizontal shaker and vigorously shaken for 40 minutes. After 40 minutes the washing solution was poured off and the embryos were briefly rinsed in H₂O.

For DNA extraction, each embryo was placed into a well of a 96 well block, to each embryo 100 μl of a 5 mM NaOH solution was added. The NaOH conc. could vary between approximately 2.5 mM and approximately 50 mM. the soaking time could vary between approximately 20 minutes and approximately 3 hours. Any other chemical which disrupts cells but does not harm the viability of the embryos could be used (e.g. Potassium Hydroxide (KOH)) or any chemical substances which do not harm the viability of the embryos could be added to a cell disrupting solution.

The 96 well block was placed on a horizontal shaker, the blocks were slowly agitated for 1 hour. The alkali solution disrupts scutellar or embryo axis cells, the DNA and other cellular content of the disrupted cells is released into the solution. After 1 hour, 80 μL of the soaking solution from each well were transferred into the wells of a second block and 1M TrisHCl (or any other pH adjusting chemical which does not harm downstream application of the DNA) was added to lower the pH to around 8. The embryos were washed several times in MS buffer containing 3% sucrose.

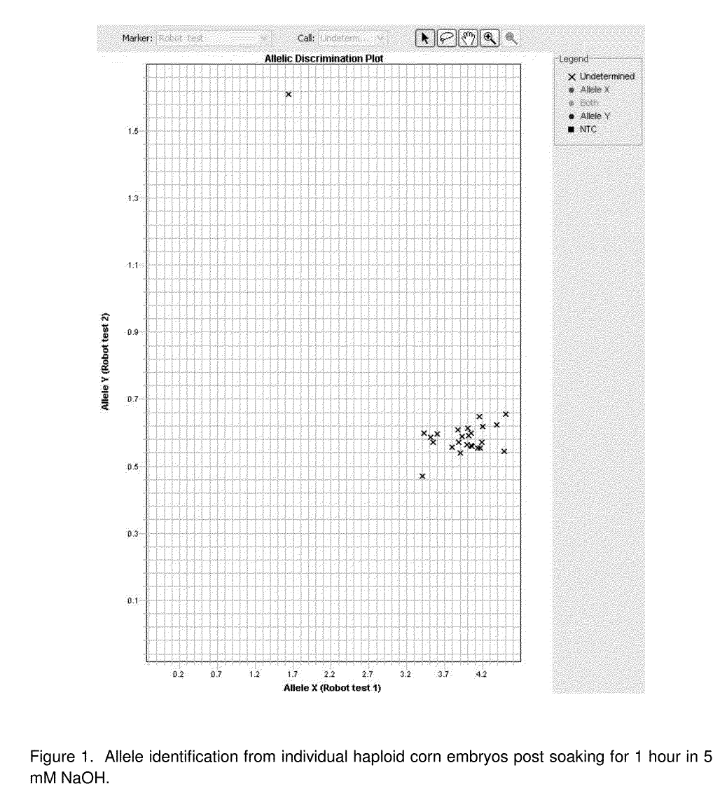

For molecular marker analysis, the DNA from 24 embryos which was released into the soaking solution was used directly as a template for Taqman PCRs. At this point, any DNA cleaning processes could be applied. Modification of the DNA (e.g. additional cleaning steps via alcohol precipitation) could be applied. Direct PCR results are shown in FIG. 1

Experiment 2.

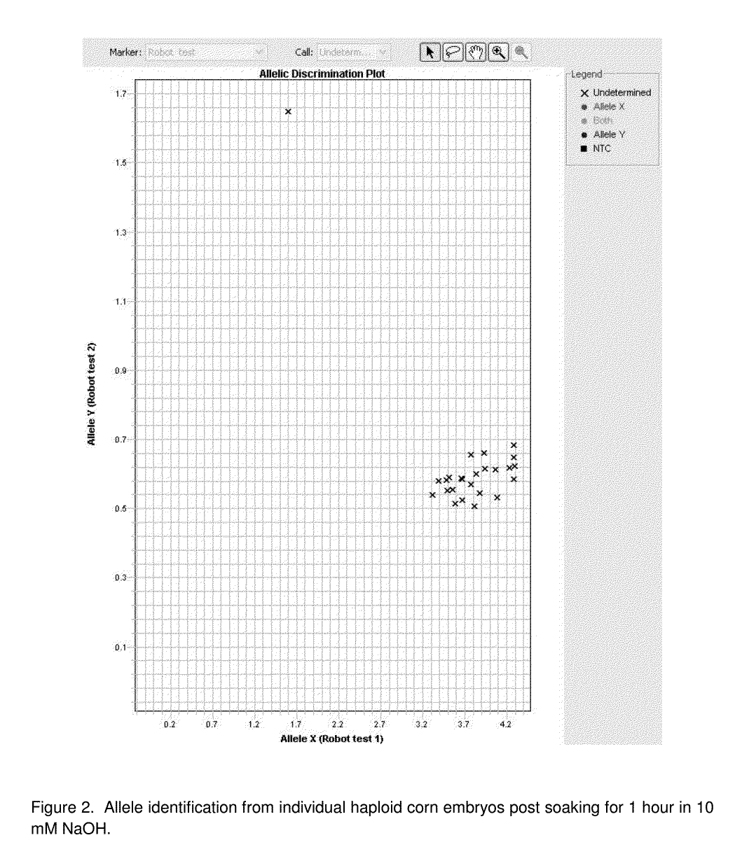

This experiment was performed as described under experiment 1, but DNA was extracted by soaking embryos in 10 mM NaOH. The result for the molecular marker analysis is shown in FIG. 2.

TABLE 1
Embryo germination rates observed from embryos
soaked in NaOH (5, 10 mM) for 1 hour.

| | DNA | Initial | Number | Percent |
|---|---|---|---|---|
| Embryo | Extraction | Number | Germinated | Embryo |
| Ploidy | Method | of Embryos | Embryos | Germination |
| Haploid | Control | 541 | 534 | 99% |
| Haploid | Embryos | 29 | 29 | 100% |
| | soaked in | | | |
| | 10 mM | | | |
| | NaOH for | | | |
| | 1 hr. | | | |
| Haploid | Embryos | 31 | 31 | 100% |
| | soaked in | | | |
| | 5 mM | | | |
| | NaOH for | | | |
| | 1 hr. | | | |

### Example 4

### Methods of Endosperm Tissue Removal from Haploid Immature Corn Embryos

Endosperm Wash Method 1.

Haploid embryos representing two different F1 hybrids were independently pooled and washed for 5 minutes in 10 mM NaOH and for an additional 15 minutes in MS buffer plus 3% sucrose. Any liquid chemical (e.g. tween 20) can be used to cleanse the embryos as long as the embryo viability is not harmed by the cleansing process. Each washing step was performed in a 50 ml tube. The tube was shaken vigorously on a horizontal shaker. After both washing steps the embryos were rinsed in H₂O.

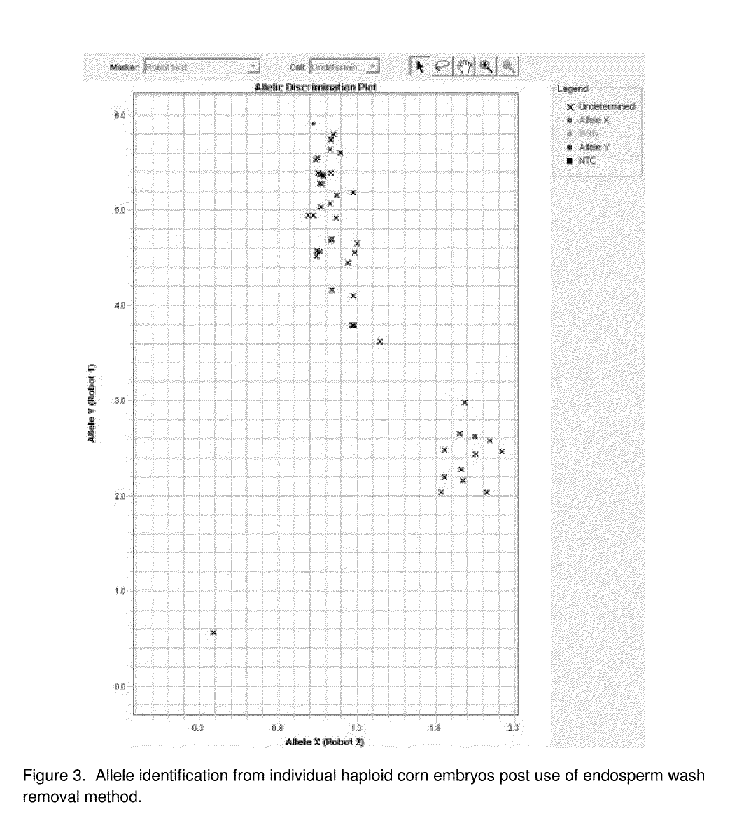

DNA extraction and marker analysis were performed as described under experiment 1, but the embryos were soaked in 10 mM NaOH. The result for the marker analysis is shown in FIG. 3.

Endosperm Wash Method 2

This endosperm washing method 2 was performed as in endosperm wash method 1 (50 ml tubes with heavy shaking), but endosperm tissue was removed by washing embryos in MS buffer plus 3% sucrose for 30 minutes.

DNA was extracted as described under endosperm wash method 1.

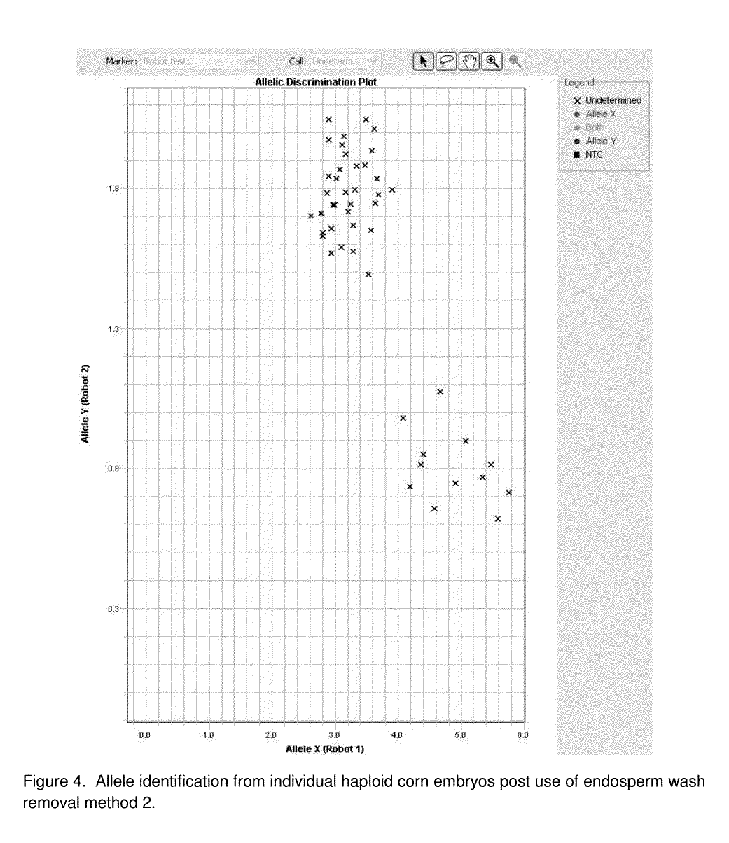

The result for the molecular marker analysis is shown in FIG. 4.

TABLE 2
Embryo germination rates observed from embryos soaked
in NaOH and using endosperm wash methods 1 & 2.

| Embryo | | | | |
|---|---|---|---|---|
| Ploidy/ | Endosperm | Initial | Number | Percent |
| Parent | Wash | Number | Germinated | Embryo |
| Source | Method | of Embryos | Embryos | Germination |
| Haploid, | Control | 42 | 36 | 86% |
| Parent1 | | | | |
| Haploid, | Control | 26 | 25 | 96% |
| Parent2 | | | | |
| Haploid, | Endosperm | 28 | 23 | 82% |
| Parent1 | Wash | | | |
| | Method 1 | | | |
| Haploid, | Endosperm | 29 | 27 | 93% |
| Genotype1 | Wash | | | |
| | Method 2 | | | |
| Haploid, | Endosperm | 38 | 38 | 100% |
| Parent2 | Wash | | | |
| | Method 1 | | | |
| Haploid, | Endosperm | 27 | 24 | 89% |
| Parent2 | Wash | | | |
| | Method 2 | | | |

### Example 5

### Soaking of Diploid Embryos in NaOH to Facilitate DNA Extraction

Experiment 5 Removal of Endosperm Tissue was Done as Described in Example 4.

Each embryo was placed into a well of a 96 well block, to each embryo 200 μl of a 20 mM NaOH solution was added. The 96 well block was placed on a horizontal shaker, the blocks were slowly agitated for 1 hour. After 1 hour, 160 μl of the NaOH solution from each well were transferred into the wells of a second block and 1M TrisHCl (or any other pH adjusting chemical which does not harm downstream application of the DNA) added to lower the pH to around 8. The embryos were washed several times in sterile water and 0.5×TE.

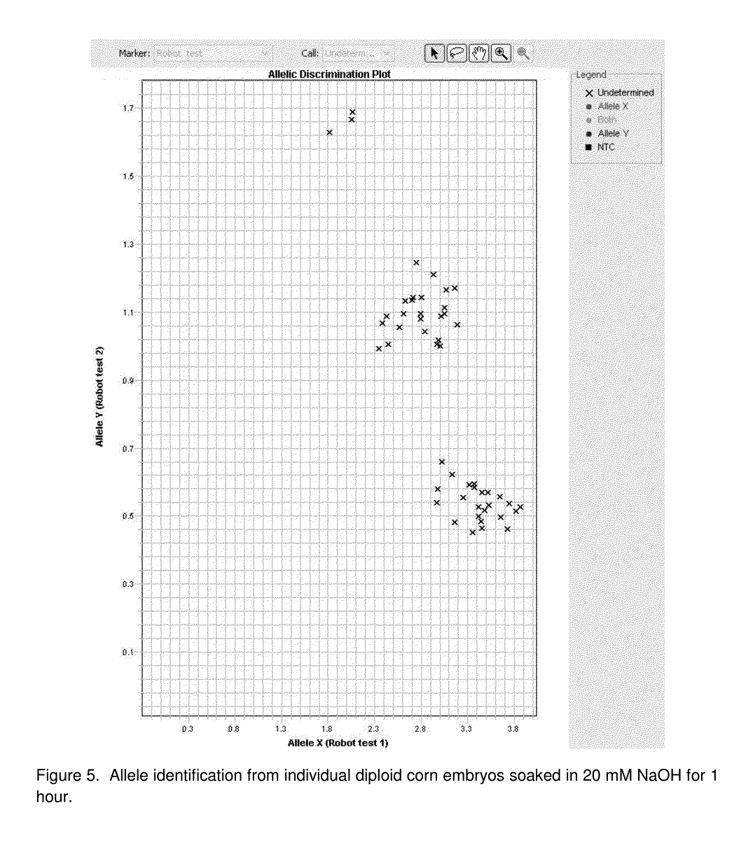

For molecular marker analysis, the DNA from 24 embryos which was released into the soaking solution was used directly as a template for Taqman PCR reaction. Results are shown in FIG. 5

TABLE 3
Embryo germination rates observed from diploid
embryos soaked in 20 mM NaOH for one hour.

| | DNA | Initial | Number | Percent |
|---|---|---|---|---|
| Embryo | Extraction | Number | Germinated | Embryo |
| Ploidy | Method | of Embryos | Embryos | Germination |
| Diploid | Control | 50 | 48 | 96% |
| Diploid | DNA | 48 | 46 | 96% |
| | Extracted | | | |
| | by | | | |
| | Soaking in | | | |
| | NaOH | | | |

### Example 6

### Removal of Immature Corn Scutellar Tissue from Haploid and Diploid Immature Corn Embryos with a Small Bore Pipette Tip

Experiment 6

Scutellar tissue from immature diploid and haploid corn embryos (approximately 4-6 mm) was sampled by penetrating the scutellum with a 2 μl pipette tip (VWR North America Cat. No. 53509-130). Scutellum tissue containing pipette tips were placed vertically into wells of 96 well blocks. To facilitate the removal of the tissue plug, 50 μl of 30 mM NaOH was added. The pipette tips remained in the NaOH solution for 1 hour. After 1 hour each tissue plug was released into the NaOH solution via positive air pressure. The plugs were kept one hour in NaOH to disrupt the cells further and release the DNA into the NaOH solution. Finally 1 M TrisHCl or any other pH adjusting chemical which does not harm downstream application of the DNA could be added to adjust the pH of the DNA containing solution to around 8. Any other cell disrupting agent (e.g KOH) could be added or the cells of the plug could be disrupted mechanically e.g. with a steel bead or a steel pin. The DNA could be used directly as template for PCR reactions or the DNA or additional cleaning steps could be applied. Germination evaluations were conducted as previously described in experiments 1 and 2.

TABLE 4
Embryo germination rates observed in diploid embryos post
scutellar tissue sampling with a 2 μl pipette tip.

| | DNA | Initial | Number | Percent |
|---|---|---|---|---|
| Embryo | Extraction | Number | Germinated | Embryo |
| Ploidy | Method | of Embryos | Embryos | Germination |
| Diploid | Control | 25 | 25 | 100% |
| Diploid | Scutellar | 25 | 25 | 100% |
| | Plug | | | |
| | Sampling | | | |

TABLE 5
Embryo germination rates observed from haploid embryos
post tissue sampling with a 2 ul pipette tip.

| | DNA | Initial | Number | Percent |
|---|---|---|---|---|
| Embryo | Extraction | Number | Germinated | Embryo |
| Ploidy | Method | of Embryos | Embryos | Germination |
| Haploid | Control | 643 | 615 | 96% |
| Haploid | Scutellar | 276 | 264 | 96% |
| | Plug | | | |
| | Sampling | | | |

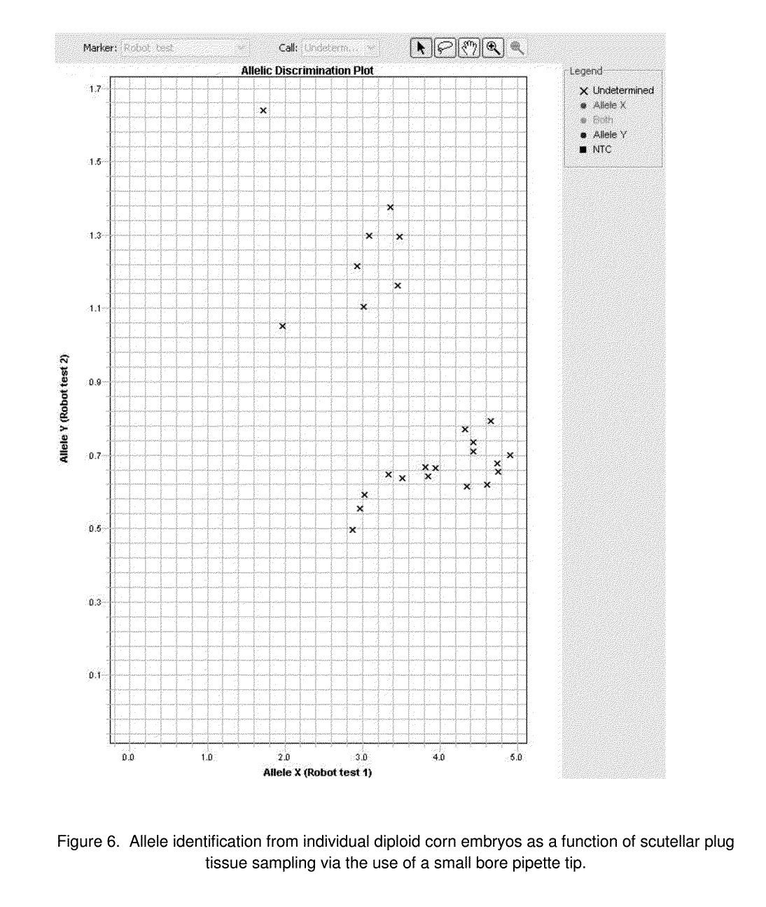

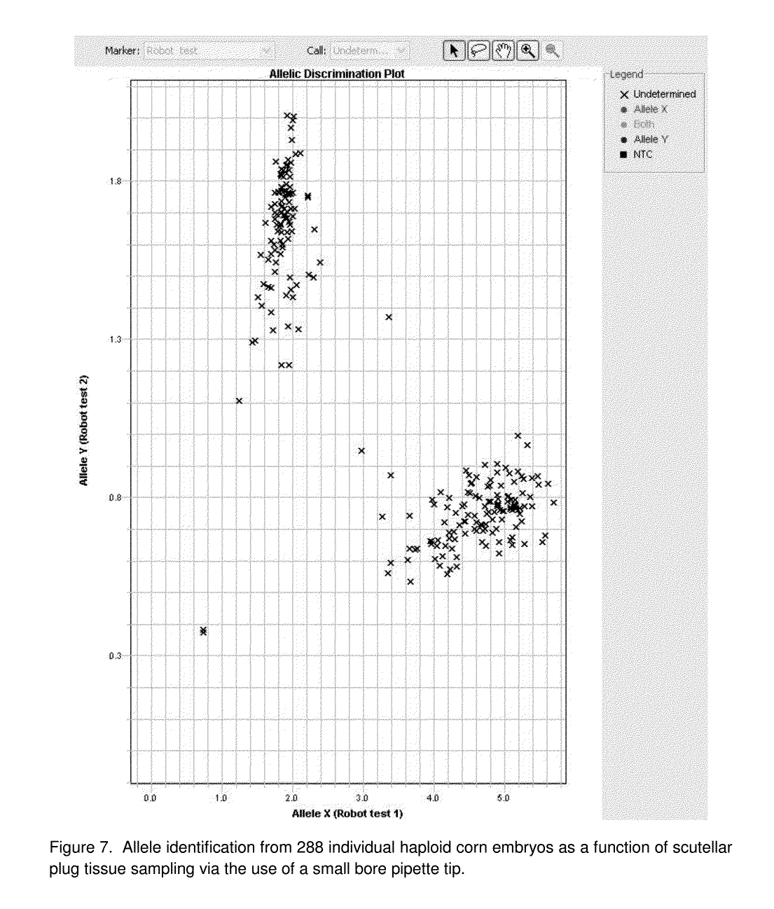

For molecular marker analysis, the DNA was used directly as a template for Taqman PCR reaction. Results for 288 haploid DNA samples are shown in FIG. 7. Results for diploid DNA samples are shown in FIG. 6.

### Example 7

### Removal of Immature Corn Scutellar Tissue from Haploid Immature Corn Embryos by Scutellum Penetration with a Sharp Tip

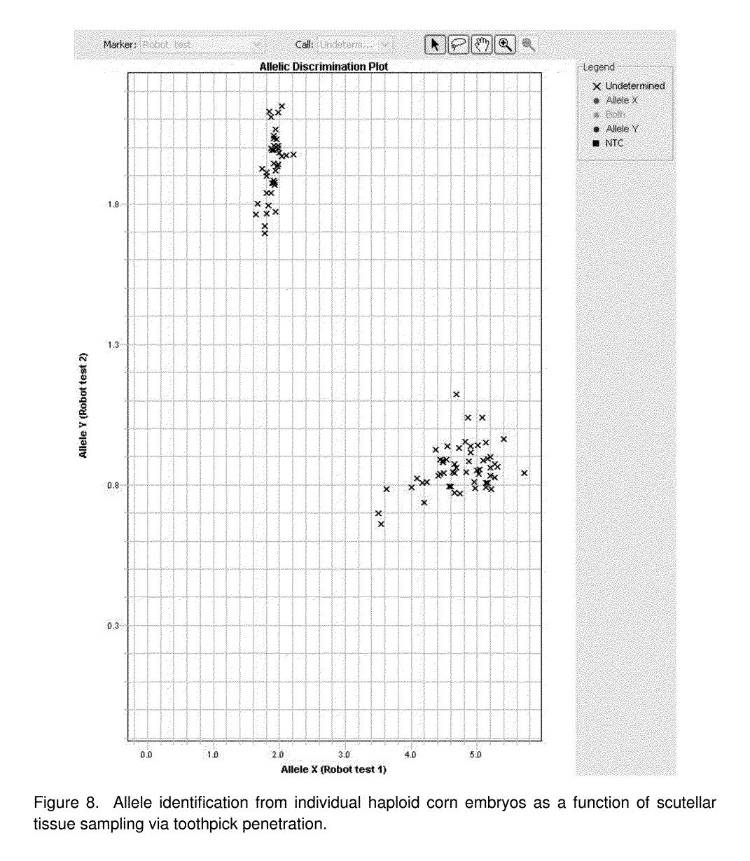

Experiment 8. Tissue/DNA extraction from haploid immature corn embryo scutellar tissue via penetration with a toothpick. Toothpick tips were sharpened to a narrow point and used to penetrate into and through the scutellar surface. The sharpened toothpick ends were then placed vertically into wells of 96 well blocks. 50 μl of 30 mM NaOH was added to each well. Any other solutions which do not interfere negatively with downstream applications of the isolated DNA could be used. The scuttelum tissue was released mechanically by rubbing the toothpick tips against the well walls. 1 M TrisHCl was added (or any other pH adjusting chemical which does not harm downstream application of the DNA) to adjust the pH of the DNA containing solution to around 8. For molecular marker analysis, the DNA was used directly as a template for Taqman PCRs. Results for 96 DNA samples are shown in FIG. 8.

TABLE 6
Embryo germination rates observed in haploid embryos
post tissue sampling by toothpick scutellar penetration.
Same control data was used in tables 5 and 6.

| | DNA | Initial | Number | Percent |
|---|---|---|---|---|
| Embryo | Extraction | Number | Germinated | Embryo |
| Ploidy | Method | of Embryos | Embryos | Germination |
| Haploid | Control | 643 | 615 | 96% |
| Haploid | Toothpick | 103 | 97 | 94% |
| | Penetration | | | |

### Example 8

### Tissue/DNA Extraction from Diploid Immature Corn Embryo Scutellar Tissue Via Penetration with a Toothpick

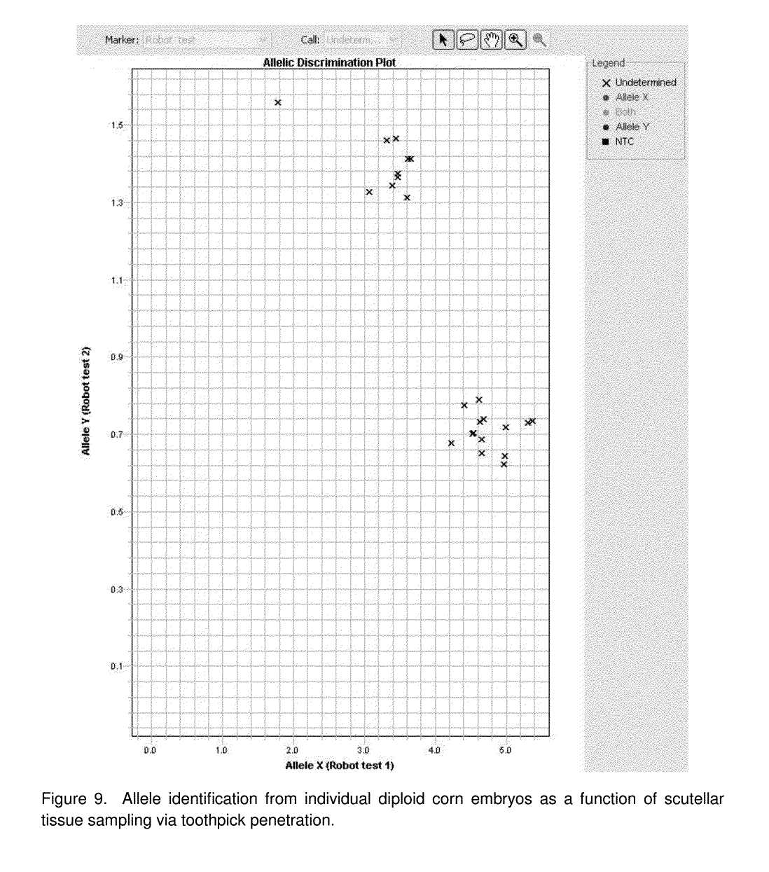

DNA Extraction: DNA extraction and molecular marker analysis from diploid embryos was done as described under experiment 8. Results for DNA samples are shown in FIG. 9

TABLE 7
Embryo germination rates observed in diploid embryos post
tissue sampling by toothpick scutellar penetration.

| | DNA | Initial | Number | Percent |
|---|---|---|---|---|
| Embryo | Extraction | Number | Germinated | Embryo |
| Ploidy | Method | of Embryos | Embryos | Germination |
| Diploids | Control | 25 | 25 | 100% |
| Diploids | Toothpick | 25 | 25 | 100% |
| | Penetration | | | |

### Example 8

### Viability Evaluation of Germinated Embryos Soaked in NaOH

Five days post germination, germinated embryos were washed in MS buffer plus 3% sucrose for 30 minutes as described in experiment 1. After 30 minutes the washing solution was poured off and 40 ml of 5 mM NaOH or 40 ml of 10 mM NaOH were added. The tubes were slowly agitated on a horizontal shaker. After 30 minutes, the embryos were removed, washed with MS +3% and the germination capacity evaluated as described under example 2. The results show that late stage germinated embryos survive NaOH soaking.

TABLE 8
Embryo germination rates observed from germinated
haploid embryos post soaking in NaOH.

| | DNA | Initial | Number | Percent |
|---|---|---|---|---|
| Embryo | Extraction | Number | Germinated | Embryo |
| Ploidy | Method | of Embryos | Embryos | Germination |
| Haploid | Germinated | 13 | 11 | 85% |
| | embryos | | | |
| | soaked | | | |
| | with 10 mM | | | |
| | NaOH | | | |
| Haploid | Germinated | 28 | 26 | 93% |
| | embryos | | | |
| | soaked | | | |
| | with 5 mM | | | |
| | NaOH | | | |

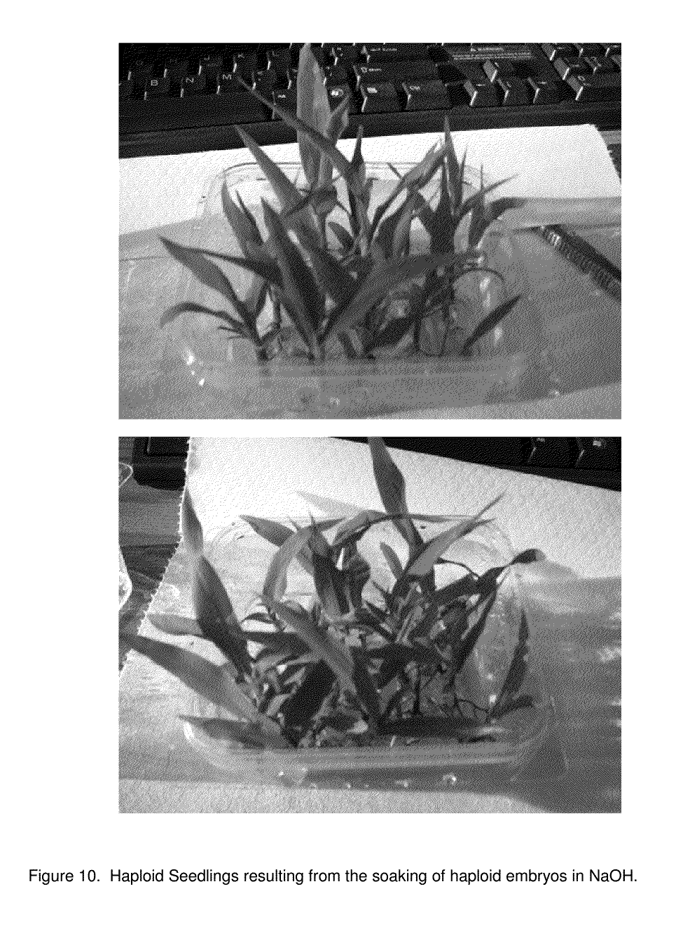

Pictures illustrating haploid seedlings derived the two NaOH treatments are found in FIG. 10.

### Example 9

### Scutellar Plug Sampling and Stasis Storage

Haploid embryos were identified 24 hours post extraction from corn kernels. Embryos were subsequently either 1) germinated on solidified MS medium or 2) tissue sampled via the collection of a scutellar tissue plug and germinated on solidified MS medium or 3) placed on an MS moistened filter paper in a petri dish which was then moved into a dark and cooled chamber (7 C/45 F) for a period of 8 days after which the embryos were germinated on solidified MS medium or 4) tissue sampled via the collection of a scutellar tissue plug and placed on an MS moistened filter paper in a petri dish which was moved into a dark cool (7 C/45 F) chamber for a period of 8 days after which the embryos were germinated on solidified MS medium. Surviving seedlings were transplanted into flats containing a Fafard potting mixture.

TABLE 9
Seedling transplantation efficacy as a function
of tissue sampling and stasis storage.

| Haploid | Initial | No. of | Percent |
|---|---|---|---|
| Embryo | No. of | Transplanted | Seedlings |
| Treatment | Embryos | Seedlings | Transplanted |
| No Cold | 50 | 35 | 70% |
| Storage | | | |
| No Cold | 55 | 35 | 64% |
| Storage | | | |
| and | | | |
| Scutellar | | | |
| Plug | | | |
| Sampling | | | |
| Stasis | 58 | 37 | 64% |
| Storage of | | | |
| Embryos | | | |
| Scutellar | 55 | 45 | 82% |
| Plug | | | |
| Sampling | | | |
| and Stasis | | | |
| Storage | | | |

1. A non-destructive, viability retaining method of sampling a plant embryo, comprising the step of removing a tissue sample or releasing and collecting cellular contents comprising at least a portion of the scutellum and/or embryonic axis region of the embryo.

2. The method of sampling a plant embryo according to claim 1, further comprising the step of performing analysis on the tissue sample or cellular contents from the embryo.

3. The method of sampling a plant embryo according to claim 2, further comprising the step of selecting an embryo based on the result of the analysis.

4. The method of sampling a plant embryo according to claim 1, wherein said method is automated or semi-automated.

5. The method according to claim 1, wherein the embryo is at the non-mature stage of development.

6. The method according to claim 1, wherein the sample removed comprises tissue from the coleoptilar end of the embryo.

7. The method according to claim 1, wherein the sample removed comprises tissue from the non-coleoptilar end of the embryo.

8. The method according to claim 1, wherein the sample removed comprises tissue from the equator or middle region of the embryo.

9. The method according to claim 1, wherein the sample is removed using a penetrant such as a toothpick, a pipette tip, or similar device.

10. The method according to claim 1, wherein the cells and/or cellular content is removed using sandpaper, or similar material to abrade and retain tissue or cellular contents.

11. The method according to claim 1, wherein the sample is removed by cell disruption digestion or by using a liquid such as sodium hydroxide solution to bathe the embryo for a sustained period of time.

12. The method according to claim 11, further comprising the collection of cells or cellular contents from the liquid.

13. The method according to claim 1, wherein the sample is removed by slicing with a knife or a laser.

14. The method according to claim 1, wherein the removed sample is subjected to genetic, chemical or biochemical analysis.

15. The method according to claim 1, wherein the embryo is a Zea mays embryo.

16. The method according to claim 1, wherein the embryo is a Triticum embryo.

17. The method according to claim 1, wherein the embryo is a Oryza embryo.

18. A plant embryo, wherein said embryo has been sampled according to the method of claim 1.

19. A method of growing a plant comprising growing a plant embryo according to claim 18 in plant production.

20. A maize plant produced from an embryo according to claim 19.

21. A method of data driven plant advancement, comprising the steps of
a. inducing haploids;
b. extracting and identifying embryos from amongst a mixed population of diploid and haploid embryos;
c. obtaining a tissue sample, cells or cellular contents from the extracted haploid embryos in a non-destructive manner, said samples preferably comprising the scutellum region; and
d. optionally, the application of a doubling chromosome process;
wherein step c) can take place before or after germination.

22. A method of data driven plant advancement according to claim 21, further comprising one or more of the steps of
e. analyzing the genetic, chemical or biochemical characteristics of the sampled diploid or haploid embryos;
f. advancing selected embryos based on the analyzed characteristics; and
g. producing seedlings from the selected embryos.

23. A method of data driven plant advancement according to claim 21 wherein chromosome doubling is not performed.

24. A method of data driven plant advancement according to claim 22, further comprising clonal propagation or by the production of seeds from the seedlings of step g).

25. A method of data driven plant enhancement according to claim 21, wherein the embryos are stored until the outcome of the genetic, chemical or biochemical analysis is known.

26. A method of data driven plant advancement according to claim 25, wherein the embryos are stored in a controlled atmosphere and temperature.

27. A method of data driven plant enhancement according to claim 21, wherein
a. the embryo is manipulated using a robotic arm;
b. a camera is used to select haploids or non-haploids; and
c. a camera or vision system is used to guide the sampling instrument to the tissue to be sampled.

28. A method of haploid embryo discrimination, comprising the steps of
a. capturing an image of a population of embryos;
b. identifying haploid embryos in said population; and
c. moving selected embryos via transporting means;

29. A method of haploid embryo discrimination according to claim 28, further comprising the step of
d. transferring all embryos or selected embryos to a different location using said transporting means.

30. A method of haploid embryo discrimination according to claim 28, further comprising the steps of
d. placing the attached embryos such that tissue sample(s) can be removed from the embryos; and
e. transferring all embryos or selected embryos to a different location.

31. A method of haploid embryo discrimination according to claim 28, further comprising the steps of
d. placing the attached embryos such that a tissue sample or cellular contents can be removed from the embryos; and
e. transferring all embryos or selected embryos to a controlled atmosphere and temperature environment suitable for embryo storage.

32. A method of haploid embryo discrimination according to claim 28, wherein the embryos are identified using algorithms based on either color or spectral analysis.
